(19) United States
(12) Patent Application Publication
(10) Pub. No.: US 2023/0233256 A1
(43) Pub. Date: Jul. 27, 2023

(54) ENERGY SOURCE FOR PRECONDITIONING AN ENERGY GUIDE IN A CATHETER SYSTEM

(71) Applicant: BOLT MEDICAL, INC., Carlsbad, CA (US)

(72) Inventors: Christopher A. Cook, Laguna Niguel, CA (US); Rachel Troutman, Carlsbad, CA (US); Brett Wingad, Vista, CA (US)

(21) Appl. No.: 18/100,784

(22) Filed: Jan. 24, 2023

Related U.S. Application Data
(60) Provisional application No. 63/303,501, filed on Jan. 27, 2022.

(51) Int. Cl. A61B 18/26; A61B 17/22

(52) U.S. Cl. CPC A61B 18/26; A61B 17/22022; A61B 2018/266; A61B 2017/22051; A61B 2018/0022

(57) ABSTRACT

A catheter system (100) for treating one or more treatment sites (106) within or adjacent to a vessel wall (108) or a heart valve includes an energy source (124), an energy guide (122A), and a plasma generator (133). The energy source (124) is configured to generate (i) an energizing pulse, and (ii) a conditioning pulse that alternately generates a lower energy than the energizing pulse. The energy source (124) includes a source adjuster (224A) that conditions the energy source (124). The energy guide (122A) is configured to selectively receive energy. The energy guide (122A) includes a guide proximal end (122P) and a guide distal end (122D). The energy source (124) is coupled to the guide proximal end (122P). The plasma generator (133) is coupled to the guide distal end (122D). The plasma generator (133) includes a generator target (233T) having a target surface (233S).

### RELATED APPLICATION

This application is related to and claims priority on U.S. Provisional Patent Application Ser. No. 63/303,501 filed on Jan. 27, 2022 and entitled “ENERGY SOURCE FOR PRECONDITIONING A PLASMA GENERATOR IN A CATHETER SYSTEM.” To the extent permissible, the contents of U.S. Provisional Application Ser. No. 63/303,501 are incorporated in its entirety herein by reference.

### BACKGROUND

Vascular lesions within vessels in the body can be associated with an increased risk for major adverse events, such as myocardial infarction, embolism, deep vein thrombosis, stroke, and the like. Severe vascular lesions can be challenging to treat and achieve patency for a physician in a clinical setting. Vascular lesions may be treated using interventions such as drug therapy, balloon angioplasty, atherectomy, stent placement, vascular graft bypass, to name a few. Such interventions may not always be ideal or may require subsequent treatment to address the lesion.

Energy-driven plasma generator devices often utilize energies capable of ionizing matter. Higher energy levels make it inherently challenging to couple the energy into and out of matter to transmit it over long distances. One issue encountered in creating a practical device is damage to the proximal end of the transmission medium. This is due to physical limitations of the material as well as interface quality and contamination. These factors limit the energy damage threshold. Damage to the bulk material and distal end also occurs. The plasma-generating mechanism creates bubbles and small particles in the medium that surrounds the plasma generator. These inclusions limit energy transmission from the energy guide to the plasma generator, reducing conversion efficiency and the overall yield of the device. These limitations can apply to any type of lithotripsy system regardless of the energy source.

### SUMMARY

The present invention is directed toward a method for conditioning one of an energy guide and a plasma generator in a catheter. In various embodiments, the method includes the steps of positioning a plasma generator within an inflatable balloon near a distal end of an energy guide; coupling a proximal end of the energy guide to an energy source; positioning the inflatable balloon adjacent to a treatment site; adjusting the energy source with a source adjuster to generate (a) a conditioning pulse having a conditioning energy level, and (b) an energizing pulse having an energizing energy level, the conditioning energy level being lower than the energizing energy level; and alternately delivering the conditioning pulse and the energizing pulse to the proximal end of the energy guide to condition one of (i) the proximal end of the energy guide, and (ii) the plasma generator, to generate a plasma within the inflatable balloon.

In some embodiments, the step of coupling includes optically coupling the proximal end of the energy guide to the energy source.

In certain embodiments, the step of coupling includes electrically coupling the proximal end of the energy guide to the energy source.

In various embodiments, the source adjuster automatically adjusts the energy source to alternate between the conditioning energy level and the energizing energy level.

In some embodiments, the source adjuster adjusts the energy source to alternate between the conditioning energy level and the energizing energy level after a predetermined number of conditioning pulses.

In certain embodiments, the catheter includes a plurality of energy guides each having a proximal end and a distal end, each distal end being positioned near a respective spaced-apart plasma generator within the inflatable balloon, and the steps of adjusting and alternately delivering are implemented for each of the plurality of energy guides.

In various embodiments, the conditioning energy level is applied to the proximal end of each of the plurality of energy guides before the energizing energy level is applied to the proximal end of each of the plurality of energy guides.

In some embodiments, the conditioning energy level and the energizing energy level are applied to the proximal end of each of the plurality of energy guides in an alternating manner.

In certain embodiments, the source adjuster alternates from the conditioning energy level to the energizing energy level after a predetermined number of conditioning pulses.

In various embodiments, the energy guide includes an optical fiber, and the energy source includes a laser.

In some embodiments, the energy guide includes a pair of metallic wires, and the energy source includes a high-voltage energy generator.

In certain embodiments, the step of positioning the plasma generator includes the plasma generator including a metallic plasma target that receives energy via the energy guide.

The present invention is also directed toward a catheter system for generating a plasma within an inflatable balloon. In certain embodiments, the catheter system includes an energy guide, an inflatable balloon, a plasma generator, and an energy source. The energy guide is configured to selectively receive energy, the energy guide including a guide proximal end and a guide distal end. The plasma generator is positioned within the balloon near the guide distal end of the energy guide. The energy source is coupled to the guide proximal end of the energy guide, the energy source including a source adjuster that is configured to alternately generate an energizing pulse and a conditioning pulse having a lower energy level than the energizing pulse, the conditioning pulse conditioning one of (a) the guide proximal end of the energy guide, and (b) the plasma generator.

In various embodiments, the energy guide is optically coupled to the energy source.

In some embodiments, the energy guide is electrically coupled to the energy source.

In certain embodiments, the source adjuster is configured to automatically alternately adjust the energy source between the conditioning energy level and the energizing energy level.

In various embodiments, the source adjuster is configured to adjust the energy source to alternate between the conditioning energy level and the energizing energy level after a predetermined number of conditioning pulses.

In some embodiments, the energy guide includes an optical fiber, and the energy source includes a laser.

In certain embodiments, the energy guide includes a pair of metallic wires, and the energy source includes a high-voltage energy generator.

In various embodiments, the plasma generator includes a metallic plasma target that receives energy via the energy guide.

This summary is an overview of some of the teachings of the present Application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic diagram of one embodiment of a portion of a catheter system having features of the present invention;

FIG. 2 is a simplified schematic diagram of one embodiment of a portion of the catheter system;

FIG. 3 is a flowchart outlining one embodiment of a method for preconditioning an energy guide within the catheter system; and

FIG. 4 is a flowchart outlining another embodiment of a method for preconditioning an energy guide within the catheter system.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DESCRIPTION

Treatment of vascular lesions (also sometimes referred to herein as “treatment sites”) can reduce major adverse events or death in affected subjects. As referred to herein, a major adverse event is one that can occur anywhere within the body due to the presence of a vascular lesion. Major adverse events can include but are not limited to, major adverse cardiac events, major adverse events in the peripheral or central vasculature, major adverse events in the brain, major adverse events in the musculature, or major adverse events in any of the internal organs.

As used herein, the terms “intravascular lesion,” “vascular lesion,” and “treatment site” are used interchangeably unless otherwise noted. The intravascular lesions and/or the vascular lesions are sometimes referred to herein simply as “lesions.” Also, as used herein, the terms “focused location” and “focused spot” can be used interchangeably unless otherwise noted and can refer to any location where the light energy is focused to a small diameter than the initial diameter of the energy source.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention, as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming. However, it would nevertheless be a routine undertaking of engineering for those of ordinary skill in the art having the benefit of this disclosure.

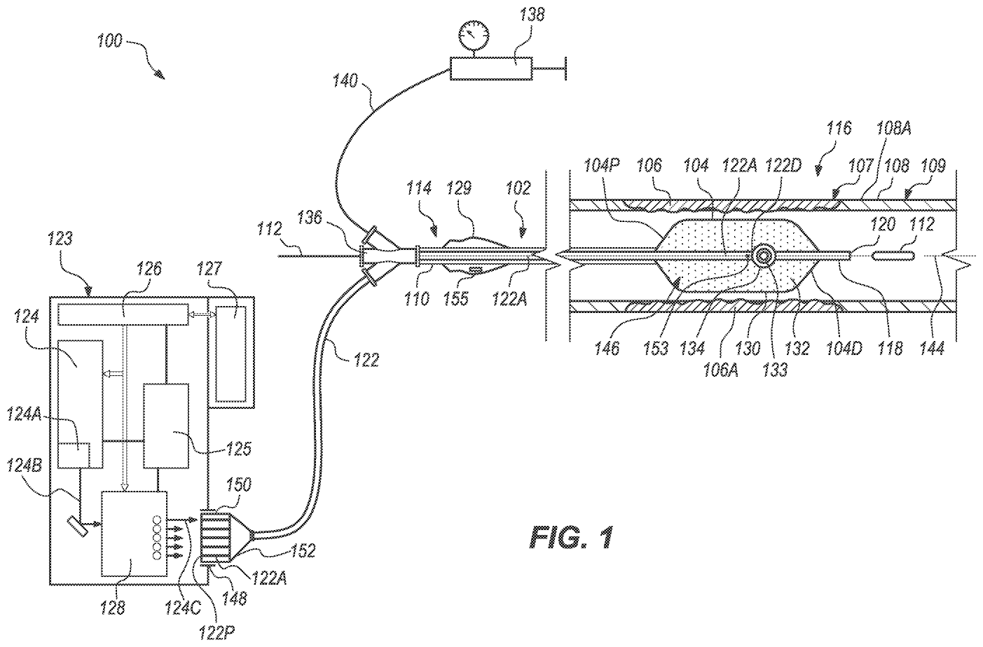

It is appreciated that the catheter systems disclosed herein can include many different forms. Referring now to FIG. 1, a schematic cross-sectional view is shown of a catheter system 100 in accordance with various embodiments herein. As described herein, the catheter system 100 is suitable for imparting pressure to induce fractures in one or more treatment sites within or adjacent to a vessel wall of a blood vessel or heart valve within a body of a patient. In the embodiment illustrated in FIG. 1, the catheter system 100 can include one or more of a catheter 102, an energy guide bundle 122 including one or more energy guides 122A, a system console 123 including one or more of an energy source 124, a power source 125, a system controller 126, a graphic user interface 127 (a “GUI”), a multiplexer 128, a source manifold 136, and a fluid pump 138. Alternatively, the catheter system 100 can include more components or fewer components than those specifically illustrated in FIG. 1.

In various embodiments, the catheter 102 is configured to move to a treatment site 106 within or adjacent to a vessel wall 108A of a blood vessel 108 within a body 107 of a patient 109. The treatment site 106 can include one or more vascular lesions 106A, such as calcified vascular lesions, for example. Additionally, or in the alternative, the treatment site 106 can include vascular lesions 106A, such as fibrous vascular lesions. Still alternatively, in some implementations, the catheter 102 can be used at a treatment site 106 within or adjacent to a heart valve within the body 107 of the patient 109.

The catheter 102 can include an inflatable balloon 104 (sometimes referred to herein simply as a “balloon”), a catheter shaft 110, and a guidewire 112. The balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter shaft 110 can also include a guidewire lumen 118, which is configured to move over the guidewire 112. As utilized herein, the guidewire lumen 118 defines a conduit through which the guidewire 112 extends. In some embodiments, the catheter 102 can have a distal end opening 120 and can accommodate and be tracked over the guidewire 112 as the catheter 102 is moved and positioned at or near the treatment site 106. In some embodiments, the balloon proximal end 104P can be coupled to the catheter shaft 110, and the balloon distal end 104D can be coupled to the guidewire lumen 118.

The balloon 104 includes a balloon wall 130 that defines a balloon interior 146. The balloon 104 can be selectively inflated with a balloon fluid 132 to expand from a deflated state, suitable for advancing the catheter 102 through a patient's vasculature, to an inflated state (as shown in FIG. 1) suitable for anchoring the balloon 104 in position relative to the treatment site 106. Stated in another manner, when the balloon 104 is in the inflated state, the balloon wall 130 of the balloon 104 is configured to be positioned substantially adjacent to and/or in contact with the treatment site 106. It is appreciated that although FIG. 1 illustrates the balloon wall 130 of the balloon 104 is shown spaced apart from the treatment site 106 of the blood vessel 108 when in the inflated state, this is done merely for ease of illustration. It is recognized that the balloon wall 130 of the balloon 104 will typically be substantially in contact with and/or directly adjacent to the treatment site 106 when the balloon 104 is in the inflated state.

The balloon 104 suitable for use in the catheter system 100 includes those that can be passed through the vasculature of a patient 109 when in the deflated state. In some embodiments, the balloon 104 is made from silicone. In other embodiments, the balloon 104 can be made from polydimethylsiloxane (PDMS), polyurethane, polymers such as PEBAX™ material, nylon, as non-exclusive examples, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In some embodiments, the balloon 104 can have a diameter (in the inflated state) ranging from at least two mm up to five mm.

In some embodiments, the balloon 104 can have a length ranging from at least three mm to 300 mm. More particularly, in some embodiments, the balloon 104 can have a length ranging from at least eight mm to 200 mm. It is appreciated that a balloon 104 having a relatively longer length can be positioned adjacent to larger treatment sites 106, and, thus, may be used for imparting pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloon 104 can include a drug-eluting coating or a drug-eluting stent structure. The drug-eluting coating or drug-eluting stent can include one or more therapeutic agents, including anti-inflammatory agents, anti-neoplastic agents, anti-angiogenic agents, and the like.

The balloon fluid 132 can be a liquid or a gas. Some examples of the balloon fluid 132 suitable for use can include, but are not limited to, one or more of water, saline, contrast medium, fluorocarbons, perfluorocarbons, gases, such as carbon dioxide, or any other suitable balloon fluid 132. In some embodiments, the balloon fluid 132 can be used as a base inflation fluid. In some embodiments, the balloon fluid 132 can include a mixture of saline to contrast medium in a volume ratio of approximately 50:50. In other embodiments, the balloon fluid 132 can include a mixture of saline to contrast medium in a volume ratio of approximately 25:75. In still other embodiments, the balloon fluid 132 can include a mixture of saline to contrast medium in a volume ratio of approximately 75:25. However, it is understood that any suitable ratio of saline to contrast medium can be used. The balloon fluid 132 can be tailored on the basis of composition, viscosity, and the like so that the rate of travel of the pressure waves are appropriately manipulated. In certain embodiments, the balloon fluid 132 suitable for use herein is biocompatible. A volume of balloon fluid 132 can be tailored by the chosen energy source 124 and the type of balloon fluid 132 used.

In some embodiments, the contrast agents used in the contrast media can include but are not limited to iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine-based contrast agents can be used. Suitable non-iodine-containing contrast agents can include gadolinium (III)-based contrast agents. Suitable fluorocarbon and perfluorocarbon agents can include, but are not limited to, agents such as perfluorocarbon dodecafluoropentane (DDFP, C5F12).

The balloon fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the balloon fluid 132 can include absorptive agents that can selectively absorb light in the mid-infrared region (e.g., at least 2.5 μm to 15 μm) or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers described herein can include neodymium:yttrium-aluminum-garnet (Nd:YAG—emission maximum=1064 nm) lasers, holmium:YAG (Ho:YAG—emission maximum=2.1 μm) lasers, or erbium:YAG (Er:YAG—emission maximum=2.94 μm) lasers. In some embodiments, the absorptive agents can be water-soluble. In other embodiments, the absorptive agents are not water-soluble. In some embodiments, the absorptive agents used in the balloon fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the one or more energy guides 122A of the energy guide bundle 122 that are in optical and/or electrical communication with the energy source 124. The energy guide(s) 122A can be disposed along the catheter shaft 110 and within the balloon 104. Each of the energy guides 122A can have a guide distal end 122D that is at any suitable longitudinal position relative to a length of the balloon 104. In some embodiments, each energy guide 122A can include an optical fiber, and the energy source 124 can be a laser. Alternatively, the energy guide 122A can include one or more electrodes, and the energy source 124 can include a high voltage generator. The energy source 124 can be in optical and or electrical communication with the energy guides 122A at the proximal portion 114 of the catheter system 100. More particularly, the energy source 124 can selectively, simultaneously, and/or sequentially be in optical and/or electrical communication with each of the energy guides 122A in any desired combination, order, and/or pattern.

In some embodiments, the catheter shaft 110 can be coupled to multiple energy guides 122A, such as a first energy guide, a second energy guide, a third energy guide, etc., which can be disposed at any suitable position about the guidewire lumen 118 and/or the catheter shaft 110. For example, in certain non-exclusive embodiments, two energy guides 122A can be spaced apart by approximately 180 degrees about the circumference of the guidewire lumen 118 and/or the catheter shaft 110; three energy guides 122A can be spaced apart by approximately 120 degrees about the circumference of the guidewire lumen 118 and/or the catheter shaft 110, or four energy guides 122A can be spaced apart by approximately 90 degrees about the circumference of the guidewire lumen 118 and/or the catheter shaft 110, etc. Still alternatively, multiple energy guides 122A need not be uniformly spaced apart from one another about the circumference of the guidewire lumen 118 and/or the catheter shaft 110. More particularly, the energy guides 122A can be disposed either uniformly or non-uniformly about the guidewire lumen 118 and/or the catheter shaft 110 to achieve the desired effect in the desired locations.

The energy guide bundle 122 can include any number of energy guides 122A in optical communication with the energy source 124 at the proximal portion 114, and with the balloon fluid 132 within the balloon interior 146 of the balloon 104 at the distal portion 116. For example, in some embodiments, the catheter system 100 and/or the energy guide bundle 122 can include from one energy guide 122A to five energy guides 122A. In other embodiments, the catheter system 100 and/or the energy guide bundle 122 can include from five energy guides 122A to fifteen energy guides 122A. In yet other embodiments, the catheter system 100 and/or the energy guide bundle 122 can include from ten energy guides 122A to thirty energy guides 122A. Alternatively, in still other embodiments, the catheter system 100 and/or the energy guide bundle 122 can include greater than 30 energy guides 122A.

The energy guide bundle 122 can also include a guide bundler 152 that brings each of the individual energy guides 122A closer together so that the energy guides 122A and/or the energy guide bundle 122 can be in a more compact form as it extends with the catheter 102 into the blood vessel 108 during use of the catheter system 100. In some embodiments, the energy guides 122A leading to the plasma generator 133 can be organized into an energy guide bundle 122, including a linear block with an array of precision holes forming a multi-channel ferrule. In other embodiments, the energy guide bundle 122 can include a mechanical connector array or block connector that organizes singular ferrules into one of (i) a linear array, (ii) a circular pattern, and (iii) a hexagonal pattern. Alternatively, the energy guide bundle 122 can include a mechanical connector array or block connector that organizes singular ferrules into another suitable array or pattern.

The energy guides 122A can have any suitable design for the purposes of generating plasma and/or pressure waves in the balloon fluid 132 within the balloon interior 146. In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide light energy along its length from a guide proximal end 122P to the guide distal end 122D, having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the length of the balloon 104 and/or relative to the length of the guidewire lumen 118 to more effectively and precisely impart pressure waves for purposes of disrupting the vascular lesions 106A at the treatment site 106.

In certain embodiments, the energy guides 122A can include one or more photoacoustic transducers 153. Each photoacoustic transducer 153 can be in optical communication with the energy guide 122A within which it is disposed. In some embodiments, the photoacoustic transducers 153 can be in optical communication with the guide distal end 122D of the energy guide 122A. Additionally, in such embodiments, the photoacoustic transducers 153 can have a shape that corresponds with and/or conforms to the guide distal end 122D of the energy guide 122A. Alternatively, no photoacoustic transducers 153 may be included.

The photoacoustic transducer 153 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 153 disposed at the guide distal end 122D of the energy guide 122A can assume the same shape as the guide distal end 122D of the energy guide 122A. For example, in certain non-exclusive embodiments, the photoacoustic transducer 153 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 153 disposed along one or more side surfaces of the length of the energy guide 122A.

In some embodiments, the energy guides 122A can further include one or more diverting features or “diverters” (not shown in FIG. 1) within the energy guide 122A that are configured to direct light to exit the energy guide 122A toward a side surface which can be located at or near the guide distal end 122D of the energy guide 122A, and toward the balloon wall 130. A diverting feature can include any system feature that diverts light energy from the energy guide 122A away from its axial path toward a side surface of the energy guide 122A. Additionally, the energy guides 122A can each include one or more light windows disposed along the longitudinal or circumferential surfaces of each energy guide 122A and in optical communication with a diverting feature. Stated in another manner, the diverting features can be configured to direct light energy in the energy guide 122A toward a side surface that is at or near the guide distal end 122D, where the side surface is in optical communication with a light window. The light windows can include a portion of the energy guide 122A that allows light energy to exit the energy guide 122A from within the energy guide 122A, such as a portion of the energy guide 122A lacking a cladding material on or about the energy guide 122A.

Examples of the diverting features suitable for use include a reflecting element, a refracting element, and a fiber diffuser. The diverting features suitable for focusing light energy away from the tip of the energy guides 122A can include but are not to be limited to, those having a convex surface, a gradient-index (GRIN) lens, and a mirror focus lens. Upon contact with the diverting feature, the light energy is diverted within the energy guide 122A to one or more of a plasma generator 133 and the photoacoustic transducer 153 that is in optical communication with a side surface of the energy guide 122A. As noted, the photoacoustic transducer 153 then converts light energy into an acoustic wave that extends away from the side surface of the energy guide 122A.

In the embodiment illustrated in FIG. 1, the system console 123 can include one or more of the light source 124, the power source 125, the system controller 126, the GUI 127, and/or the multiplexer 128. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, the GUI 127, and the multiplexer 128 can be included within the catheter system 100 without the specific need for the system console 123.

Additionally, as shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148, via which the energy guide bundle 122 is mechanically coupled to the system console 123. In such embodiments, the energy guide bundle 122 can include a guide coupling housing 150 (also sometimes referred to herein as a “ferrule”) that houses a portion, e.g., the guide proximal end 122P, of each of the energy guides 122A. The guide coupling housing 150 is configured to fit and be selectively retained within the console connection aperture 148 to provide the desired mechanical coupling between the energy guide bundle 122 and the system console 123.

The energy source 124 can be selectively and/or alternatively coupled in optical communication with each of the energy guides 122A, i.e., to the guide proximal end 122P of each of the energy guides 122A, in the energy guide bundle 122 so that the guide proximal end 122P is electrically and/or optically coupled to the energy source 124. In particular, the energy source 124 can be configured to generate energy (such as light energy, in certain embodiments) in the form of a source beam 124B, such as a pulsed source beam, that can be selectively and/or alternatively directed to and received by each of the energy guides 122A in the energy guide bundle 122 as an individual guide beam 124C. In certain embodiments, the energy emitted by the energy source 124, such as the source beam 124B and the individual guide beam 124C, can be concentrated to transmit through a long, narrow medium (such as the energy guides 122A) to bring it from the energy source 124 to the plasma generator 133 so that the plasma generator 133 can generate highly localized mechanical effects to treat localized lesions (e.g., the vascular lesion 106A). The various embodiments of the catheter system 100 described herein have improved efficacy for use in narrow human anatomy such as blood vessels 108.

Alternatively, the catheter system 100 can include more than one energy source 124. For example, in one non-exclusive alternative embodiment, the catheter system 100 can include a separate energy source 124 for each energy guide 122A in the energy guide bundle 122. The energy source 124 can be operated at low energies. In other embodiments, the energy source 124 can be electrical, such as a high-voltage pulse generator.

The energy source 124 can have any suitable design. In certain embodiments, the energy source 124 can be configured to provide sub-millisecond pulses of light energy from the energy source 124 that are focused onto a small spot in order to couple it into the guide proximal end 122P of the energy guide 122A. Such pulses of light energy are then directed and/or guided along the energy guides 122A to a location within the balloon interior 146 of the balloon 104, thereby inducing plasma formation in the balloon fluid 132 within the balloon interior 146 of the balloon 104, such as via the plasma generator 133 that can be located at the guide distal end 122D of the energy guide 122A. By locating the plasma generator 133 at the guide distal end 122D, the plasma generator 133 can be routed through torturous human anatomy, such as a constricted artery, for placement adjacent to the treatment site 106. In some embodiments, the plasma generator 133 can be at least partially formed by a metal. In various embodiments, the plasma generator 133 can be configured to be immersed in a liquid (e.g., the balloon fluid 132) that converts the plasma into a mechanical acoustic bubble and transports this energy to the treatment site 106.

In particular, the light emitted at the guide distal end 122D of the energy guide 122A energizes the plasma generator 133 to form the plasma within the balloon fluid 132 within the balloon interior 146. The plasma formation causes rapid bubble formation and imparts pressure waves upon the treatment site 106. An exemplary plasma-induced bubble 134 is illustrated in FIG. 1.

In various non-exclusive alternative embodiments, sub-millisecond pulses of light energy from the energy source 124 can be delivered to the treatment site 106 at a frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of light energy can be delivered to the treatment site 106 at a frequency that can be greater than 5000 Hz or less than one Hz or any other suitable range of frequencies.

It is appreciated that although the energy source 124 can be utilized to provide pulses of light energy, the energy source 124 can still be described as providing a single source beam 124B, i.e., a single pulsed source beam, or another suitable type of energy source such as a high voltage pulse generator.

Some energy sources 124 suitable for use can include various types of energy sources, including lasers, seed sources, and lamps. For example, in certain non-exclusive embodiments, the energy source 124 can be an infrared laser that emits light energy in the form of pulses of infrared light. Alternatively, as noted above, the energy sources 124, as referred to herein, can include any other suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths, and energy levels that can be employed to achieve plasma in the balloon fluid 132 of the catheter 102. In various non-exclusive alternative embodiments, the pulse widths can include those falling within a range, including at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter system 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 kHz. In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-alum inum-garnet (Er:YAG) laser, excimer laser, helium-neon laser, carbon dioxide laser, as well as doped, pulsed, fiber lasers. In still further embodiments, the energy source 124 can include SLEDs that have bandwidths ranging from 13.25 GHz to 18.25 GHz at 1064 nm.

The catheter system 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In various non-exclusive alternative embodiments, the catheter system 100 can generate pressure waves having maximum pressures in the range of at least approximately two MPa to 50 MPa, at least approximately two MPa to 30 MPa, or at least approximately 15 MPa to 25 MPa.

The pressure waves can be imparted upon the treatment site 106 from a distance within a range from at least approximately 0.1 millimeters (mm) to greater than approximately 25 mm, extending radially from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to 30 MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

As provided in greater detail herein, the energy source 124 can include a source adjuster 124A. The source adjuster 124A adjusts and/or conditions the energy level produced by the energy source 124 to increase the effectiveness of the catheter system 100.

The power source 125 is electrically coupled to and is configured to provide the necessary power to each of the energy source 124, the system controller 126, the GUI 127, and the handle assembly 129. The power source 125 can have any suitable design for such purposes.

The system controller 126 is electrically coupled to and receives power from the power source 125. Additionally, the system controller 126 is coupled to and is configured to control the operation of each of the energy sources 124, and the GUI 127. The system controller 126 can include one or more processors or circuits for purposes of controlling the operation of at least the energy source 124, and the GUI 127. For example, the system controller 126 can control the energy source 124 for generating pulses of light energy as desired and/or at any desired firing rate.

The system controller 126 can further be configured to control the operation of other components of the catheter system 100, such as the positioning of the catheter 102 adjacent to the treatment site 106, the inflation of the balloon 104 with the balloon fluid 132, etc. Further, or in the alternative, the catheter system 100 can include one or more additional controllers that can be positioned in any suitable manner for the purposes of controlling the various operations of the catheter system 100. For example, in certain embodiments, an additional controller and/or a portion of the system controller 126 can be positioned and/or incorporated within the handle assembly 129.

The GUI 127 is accessible by the user or operator of the catheter system 100. Additionally, the GUI 127 is electrically connected to the system controller 126. With such a design, the GUI 127 can be used by the user or operator to ensure that the catheter system 100 is effectively utilized to impart pressure onto and induce fractures at the treatment site(s) 106. The GUI 127 can provide the user or operator with information that can be used before, during, and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during the use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications, and/or desires of the user or operator.

In some embodiments, the multiplexer 128 can include a two-channel splitter design. The guide bundle 122 can include a manual positioning mechanism that is mounted on an optical breadboard and/or platen. This design enables linear positional adjustment and array tilting by rotating about a Channel 1 energy guide 122A axis (not shown in FIG. 1). The adjustment method, in other embodiments, can include at least two adjustment steps, 1) aligning the planar positions of the source beam 124B at Channel 1, and 2) adjusting the energy guide bundle 122 to achieve the best alignment at Channel 10.

As shown in FIG. 1, the handle assembly 129 can be positioned at or near the proximal portion 114 of the catheter system 100, and/or near the source manifold 136. In this embodiment, the handle assembly 129 is coupled to the balloon 104 and is positioned spaced apart from the balloon 104. Alternatively, the handle assembly 129 can be positioned at another suitable location.

The handle assembly 129 is handled and used by the user or operator to operate, position, and control the catheter 102. The design and specific features of the handle assembly 129 can vary to suit the design requirements of the catheter system 100. In the embodiment illustrated in FIG. 1, the handle assembly 129 is separate from, but in electrical, optical, and/or fluid communication with one or more of the system controller 126, the energy source 124, the fluid pump 138, and the GUI 127. In some embodiments, the handle assembly 129 can integrate and/or include at least a portion of the system controller 126 within an interior of the handle assembly 129. For example, as shown, in certain such embodiments, the handle assembly 129 can include circuitry 155 that can form at least a portion of the system controller 126.

In one embodiment, the circuitry 155 can include a printed circuit board having one or more integrated circuits, or any other suitable circuitry. In an alternative embodiment, the circuitry 155 can be omitted or can be included within the system controller 126 or otherwise within the system console 123, which in various embodiments can be positioned outside of the handle assembly 129.

The source manifold 136 can be positioned at or near the proximal portion 114 of the catheter system 100. The source manifold 136 can include one or more proximal end openings that can receive the one or more energy guides 122A of the energy guide bundle 122, the guidewire 112, and/or an inflation conduit 140 that is coupled in fluid communication with the fluid pump 138.

The fluid pump 138 that configured to inflate the balloon 104 with the balloon fluid 132 as needed.

As with all embodiments illustrated and described herein, various structures may be omitted from the figures for clarity and ease of understanding. Further, the figures may include certain structures that can be omitted without deviating from the intent and scope of the invention.

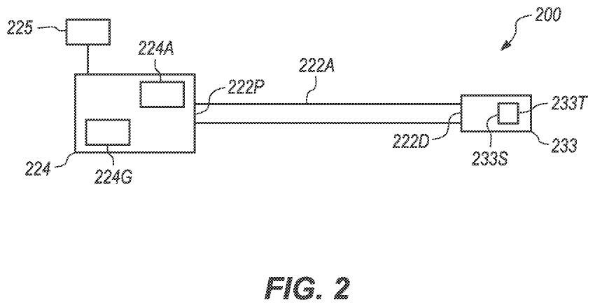

FIG. 2 is a simplified schematic diagram of another embodiment of a portion of the catheter system 200. As illustrated in FIG. 2, the catheter system 200 can include one or more energy guides 222A (only one energy guide 222A is shown in FIG. 2 for ease of understanding) having a guide distal end 222D and a guide proximal end 222P, an energy source 224, a power source 225 and a plasma generator 233.

In some embodiments, the energy guide 222A can be substituted with any suitable energy transmission medium, including metallic wiring having one or more electrodes. In certain embodiments, the energy source 224 can generate conductive electrons that are configured to transmit along and/or through the energy guide 222A. The energy source 224 can be configured to generate energy levels that (i) ionize matter, and/or (ii) work in cooperation with the plasma generator 233 to generate a plasma within the balloon 104 (illustrated in FIG. 1).

The energy source 224 can provide two or more different pulses of energy. In various embodiments, the energy source 224 can provide at least an energizing pulse and a conditioning pulse by utilizing an energy adjuster 224A. The energizing pulse results in an energizing energy level, and the conditioning pulse results in a conditioning energy level. In some such embodiments, the energizing energy level is greater than the conditioning energy level. The energizing energy level is used during the actual disruption of calcification at the treatment site 106 (illustrated in FIG. 1) and can also be referred to as “full energy.” The energizing energy level can be assumed to represent a 100% energy level, while the conditioning energy level represents less than 100% of the energizing energy level. To illustrate one simplified example, the conditioning energy level could be a 50% energy level (relative to the energizing energy level), although it is understood that this energy level is provided merely for ease of understanding and is not intended to be limiting in any manner.

In various embodiments, the energy source 224 includes the energy adjuster 224A that adjusts, conditions, reduces, and/or increases the energy level of the energy source 224. In various embodiments, by using the energy adjuster 224A, the conditioning energy level is applied by the energy source 224 to the guide proximal end 222P of the energy guide 222A prior to applying the energizing energy level to the guide proximal end 222P of the energy guide 222A. In various embodiments, the conditioning energy level can be applied by the energy source 224 to the guide proximal end 222P of the energy guide 222A less than 1 ns prior to the energizing energy level being applied to the guide proximal end 222P of the energy guide 222A. In non-exclusive alternative embodiments, the conditioning energy level can be applied by the energy source 224 to the guide proximal end 222P of the energy guide 222A less than 1 μs, less than 1 ms, less than 10 ms, less than 100 ms, less than 1 s, less than 2 s, less than 5 s, less than 10 s, and/or less than 1 minute prior to the energizing energy level being applied to the guide proximal end 222P of the energy guide 222A.

Further, it is recognized that there may be two or more different conditioning pulses that can each generate the same or different energy levels that are each lower than the energizing energy level. By way of example, and not by limitation, the energizing energy level can represent a 100% energy level, a first conditioning energy level can represent a 75% energy level (relative to the energizing energy level), and a second conditioning energy level can represent a 50% energy level (relative to the energizing energy level). It is understood that greater than two conditioning energy levels can be used.

As used herein, the term “conditioning pulse” can include one or more energy pulses having varying energy levels that are lower than the energizing energy level. It is further understood that the percentages for each conditioning pulse can vary as desired. For example, the conditioning energy level(s) can be greater or less than 50% of the energizing energy level, and can range at any energy level between 0.001% and 99.999% of the energizing energy level.

In certain embodiments, the system controller 126 (illustrated in FIG. 1) can be configured to control the sequencing of the various energy levels provided depending upon, without limitation, the number of energy guides 222A, the number of energy sources 224, the specific needs of the patient 109 (illustrated in FIG. 1), the frequency of the energy pulses, the conditioning needs of (i) the guide proximal end 222P, (ii) the guide distal end 222D, (iii) the plasma generator 233, and/or (iv) the balloon, etc., as non-exclusive examples.

In certain embodiments, the energy source 224 can include a pulse generator 224G. The pulse generator 224G can generate one or more energy pulses, which can include either or both of the energizing pulse and/or the conditioning pulse. In some embodiments, the source adjuster 224A conditions and/or adjusts the energy source 224 so that the energy source 224 generates a conditioning pulse that has a lower energy level than the energizing pulse of the energy source 224. In other embodiments, the source adjuster 224A can adjust the energy source 224 so that it generates the energizing pulse having the energizing energy level that is higher than the conditioning energy level.

The energizing pulse(s) and/or the one or more conditioning pulses can each include a single pulse and/or a plurality of pulses. In certain embodiments, the conditioning pulse can be configured to at least (i) displace inclusions in the balloon fluid 132 (illustrated in FIG. 1) surrounding the plasma generator 233, and/or (ii) condition the guide proximal end 222P of the energy guide(s) 222A. In one embodiment, the source adjuster 224A can include a separate laser (or other suitable energy sources) that provides a lower energy level than the energizing pulse level of the energy source 224. However, it is understood that both the energizing pulse and the conditioning pulse can be generated from the same energy source 224.

In some embodiments, the source adjuster 224A can be combined as a subsystem of the pulse generator 224G. In certain embodiments, the source adjuster 224A can be implemented as a control or subfunction of the pulse generator 224G. In other embodiments, the pulse generator 224G can include the source adjuster 224A. In various embodiments, the source adjuster 224A can include the pulse generator 224G.

In certain embodiments, the source adjuster 224A and the pulse generator 224G can be included within the same energy source 224. In some such embodiments, by using the source adjuster 224A the output of the energy source 224 can be at varying energy levels (at least at the conditioning energy level and the energizing energy level). The same energy source 224 can condition the guide proximal end 222P and/or the guide distal end 222D of the energy guide 222A.

In certain embodiments, the source adjuster 224A can adjust the energy output of a first laser, and the pulse generator 224G can be included in the first laser and/or a second laser. The first laser and the second laser can have the same or different wavelengths. In one non-limiting, non-exclusive example, the energy source 224 can include a UV laser light configured to condition the guide proximal end 222P and/or the guide distal end 222D of the energy guide 222. In various embodiments, the energy source 224 can include an ultrasound source and/or an electron emitter that is configured to condition the guide proximal end 222P and/or the guide distal end 222D of the energy guide 222.

In some embodiments, the energy source 224A can include electrical and/or acoustic conditioners. In one non-limiting, non-exclusive example, the source adjuster 224A can be configured to send an ultrasound pulse through the energy guide 222 to dislodge microbubbles and/or debris that may interfere with the plasma generator 233. In other embodiments, the energy source 224 can include an ultrasound transducer that is configured to oscillate and/or dislodge microbubbles and/or debris at the guide distal end 222D and/or at the plasma generator 233.

The plasma generator 233 can include a generator target 233T having a target surface 233S. The generator target 233T can be at least partially formed from a metal. In some embodiments, the energy source 224 can include a high-voltage pulse generator, the energy guide(s) 222A can include metallic wires, and the plasma generator 233 can include a spark gap. The conditioning pulse generated by the pulse generator 224G can displace and/or dislodge microbubbles that can be coupled to the target surface 233S. Additionally, the conditioning pulse can clear the generator target 233T and/or the guide proximal end 222P from contaminates. The plasma generator 233 can be spaced-apart from the guide distal end 222D within the balloon 104 (illustrated in FIG. 1).

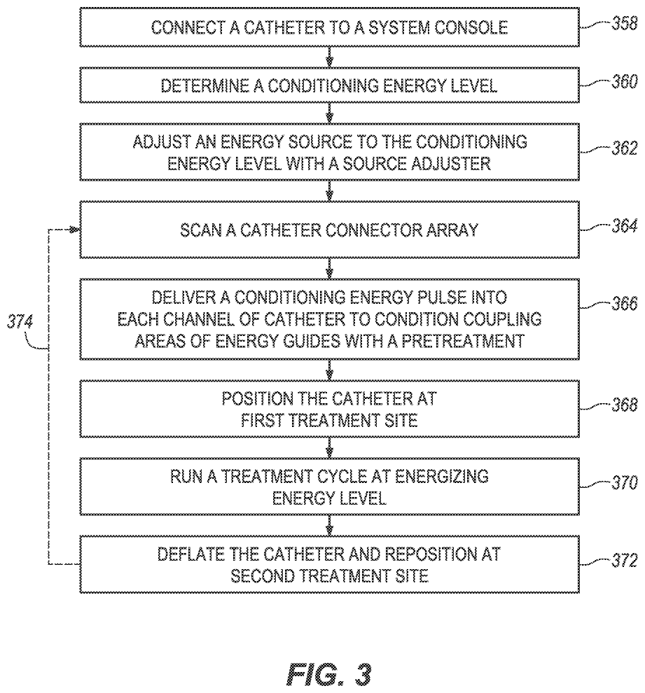

FIG. 3 is a flowchart outlining one embodiment of a method for preconditioning an energy guide and/or a plasma generator within a catheter system for use during treatment of a treatment site within or adjacent to a vessel wall or a heart valve. The method can include one or more of the following steps provided herein. It is understood that the method can include additional steps than those specifically shown and/or described herein. Additionally, or alternatively, the method can omit one or more of the steps that are specifically shown and/or described herein. Further, it is understood that the steps can be completed in any order, and the sequence of steps shown in FIG. 3 is merely provided for illustrative purposes as one non-exclusive embodiment.

The method for preconditioning an energy guide and/or a plasma generator within a catheter system can be implemented on the catheter system 100 (illustrated in FIG. 1) or other suitable systems and subsystems not explicitly shown and/or described herein. For example, the method displayed in FIG. 3 can be applied to any suitable lithotripsy system that uses an energy-driven plasma generator.

At step 358, a catheter is connected to a system console. In one embodiment, the system can start up from a standby mode at this step or at step 360. In various, non-exclusive, non-limiting embodiments, the connection step can include (i) positioning a plasma generator within an inflatable balloon near a distal end of an energy guide, (ii) coupling a proximal end of the energy guide to an energy source, and (iii) positioning the inflatable balloon adjacent to a treatment site.

At step 360, a conditioning energy level for catheter operation is determined. This determination can be predetermined and/or can be determined by an operator and/or by the system controller of the catheter system. In various embodiments, the conditioning energy level can have a lower energy level than an energizing energy level and/or a treatment energy level. The energizing energy level can be considered a nominal energy level for the nominal operation of the catheter during treatment of the treatment site.

At step 362, an energy source of the catheter system is adjusted to the conditioning energy level with a source adjuster. In various embodiments, the conditioning energy level can be a predetermined fraction of the nominal energy level (the energizing energy level).

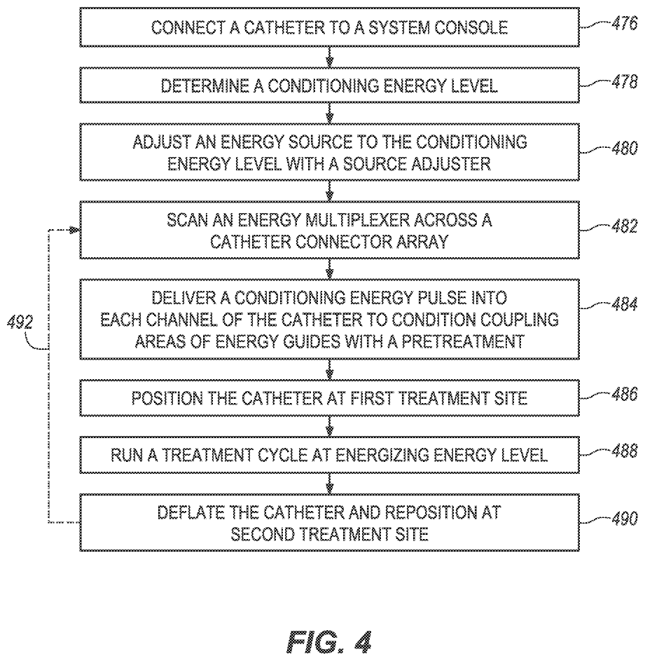

At step 364, a catheter connector array is scanned. In some embodiments, such as in step 482 (illustrated in FIG. 4), an energy multiplexer can be scanned across a catheter connector array. The catheter connector array can be scanned by any suitable scanner known in the art.

At step 366, the source adjuster can be automatically used at some point in time prior to the energizing energy level being applied, and the conditioning energy level is applied utilizing the source adjuster. In other words, the source adjuster can automatically adjust the energy source to alternate between the conditioning energy level and the energizing energy level. This automatic adjustment between the conditioning energy level and the energizing energy level can be made after a predetermined number of conditioning pulses.

Alternatively, the source adjuster can be manually initiated by an operator. The energy source delivers energy, which is adjusted by the source adjuster. An energy pulse is sequentially fired into each channel of the catheter. The energy pulse results in energy being delivered equal to the conditioning energy level. In some embodiments, the energy pulse can include one energy pulse or a sequence of energy pulses between greater than 0% of the energizing energy level and less than 100% of the energizing energy level. In various non-exclusive embodiments, the energy pulse can be at least 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99% or less than 99.999% of the energizing energy level or the treatment level.

In some embodiments, the conditioning pulse can be delivered immediately before the energizing pulse instead of firing all low-energy pulses in one pass before the energizing pulse. In such embodiments, the conditioning energy pulse can precede each energizing pulse. In some embodiments, the conditioning energy pulse can be fired with or without inflating the catheter to the inflated state to condition the proximal and/or distal end of one or more of the energy guides. The methods and systems described herein can utilize any suitable combination of energy pulse sequencing options to achieve improved conversion efficiency and overall device performance.

The conditioning pulse or pulses could be fired before or after the energizing pulse. The conditioning pulse could be a low-energy pulse at the end of the energizing pulse that could condition the plasma generator after firing the energizing pulse. The conditioning pulse step can include a continuous chain of small pulses that continuously condition the guide proximal end, the guide distal end, and/or the plasma generator. The energizing pulse can also or alternatively be fired intermittently between each sequence of small conditioning pulses.

A plurality of coupling areas of a plurality of energy guides can be conditioned with a pretreatment (the conditioning energy level). In some embodiments, when the energy source is a laser, and the energy guide is an optical fiber, the conditioning energy pulse can clear the proximal guide end of the optical fiber from various types of contamination. The conditioning energy pulse or pulses can reduce contamination or particles on the end face of the proximal guide end without damaging the surface of the proximal guide end (e.g., without damaging a glass surface of the energy guide).

The conditioning energy pulse can also reduce the likelihood that contamination vaporizing under high energy would create localized damage that, in turn, could form a pit or a localized defect that would concentrate energy and increase damage. The conditioning energy pulse can further reduce or eliminate the effects of surface contamination, thereby considerably increasing the damage threshold for a lithotripsy device. The conditioning energy pulse can also improve total energy transmission and increase the overall yield of the lithotripsy device.

At or near the distal end of the energy guide(s), the conditioning energy pulse can dislodge or displace micro-bubbles that adhere to the surface of the plasma generator target and limit the energy transmitted onto this surface which reduces the conversion efficiency. In the embodiments where the energy source is a laser, and the energy guide is an optical fiber, the low-energy pulse clears the target from bubbles or other adhering contamination. This approach could also displace other inclusions in the fluid surrounding the plasma generator, which interfere with the transmission of the energy pulse to the plasma generator. These inclusions scatter or absorb energy, thereby limiting conversion efficiency and yield. The conditioning energy pulse would minimize the impact on the plasma-generating mechanism. The conditioning pulse can be fired just before the energy pulse or at any time in between sequential pulses to prevent the buildup of inclusions in the fluid around the plasma generator or on its surfaces.

In some embodiments, the conducted energy is electrons, the energy source is a high-voltage pulse generator, the energy guide is a pair of metallic wires, and the plasma generator is a spark gap located at the distal end of the two wires. The plasma generator can also include a metallic plasma target that receives energy from the energy guide.

The conditioning step can involve firing a low-energy electrical pulse into each plasma circuit to precondition the electrical interface from the console to the wiring. This can reduce oxides and contamination, thereby improving electrical conductivity and energy transmission to the distal plasma generator. The spark gap plasma generators can suffer from the same performance issues as optical plasma generators due to micro-bubbles, particulates, and inclusions. The intermediate conditioning step between treatment cycles would be used to clear these from the spark gap, increasing conductivity, thereby improving the device's conversion efficiency and yield.

At step 368, the balloon of the catheter is positioned and pressurized at a first treatment site. An operator can position the catheter in any suitable location within the patient, including the first treatment site. As used herein, the “first,” “second,” and “third” treatment sites are merely demonstrative for ease of understanding. It is understood that the first treatment site, the second treatment site, and the third treatment site can be the same treatment site or different treatment sites. The surgeon or clinician can position the catheter at any suitable number of treatment sites based on the individual patient's treatment needs. The balloon can also be pressurized with a plasma-generating fluid to prepare the catheter system for treatment.

At step 370, one or more treatment cycles are run at the energizing energy level (also sometimes referred to herein as “full energy”). The catheter system can scan the energy multiplexer across the connector array, firing the energizing energy pulse into each channel through the entire sequence that comprises a treatment cycle. Alternatively, the catheter system can alternate between the conditioning energy level and the energizing energy level for each energy guide or for less than all energy guides.

At step 372, the balloon of the catheter can be deflated and repositioned at a second a treatment site. The catheter can then be re-inflated at the second treatment site, and the same or modified sequence of firing can occur. In some embodiments, the system can scan across the catheter connector array, firing a conditioning pulse sequentially into each catheter channel to dislodge and displace micro-bubbles and other particles. This process preconditions each energy guide and/or plasma generator for improved performance. The catheter system can then be rerun at full energy for one or more treatment cycles at the energizing energy level.

At step 374, depending on the treatment stage and/or treatment needs of the individual patient, the method can repeat steps 364 through 372 until the treatment is complete. Additionally, depending on the conditioning needs of the light guide, the interior of the inflatable balloon, and/or the plasma generator, steps 364 and 366 can be repeated until the conditioning of each of the light guide, the interior of the inflatable balloon, and/or the plasma generator is complete.

FIG. 4 is a flowchart outlining one embodiment of a method for preconditioning an energy source within a catheter system for use during treatment of a treatment site. The method can include one or more of the following steps provided herein. It is understood that the method can include additional steps than those specifically shown and/or described herein. Additionally, or alternatively, the method can omit one or more of the steps that are specifically shown and/or described herein. Further, it is understood that the method can be completed in any order, and the order of steps shown in FIG. 4 is merely provided for illustrative purposes.

The method for preconditioning an energy guide and/or a plasma generator within a catheter system can be implemented on the catheter system 100 (illustrated in FIG. 1) or other suitable systems and subsystems not explicitly shown and/or described herein. For example, the method displayed in FIG. 3 can be applied to any suitable lithotripsy system that uses an energy-driven plasma generator.

At step 476, a catheter is connected to a system console. In one embodiment, the system can start up from a standby mode at this step or at step 78. In various, non-exclusive, non-limiting embodiments, the connection step can include (i) positioning a plasma generator within an inflatable balloon near a distal end of an energy guide, (ii) coupling a proximal end of the energy guide to an energy source, and (iii) positioning the inflatable balloon adjacent to a treatment site.

At step 478, a conditioning energy level for catheter operation is determined. This determination can be predetermined and/or can be determined by an operator and/or by the system controller of the catheter system. In various embodiments, the conditioning energy level can have a lower energy level than an energizing energy level and/or a treatment energy level. The energizing energy level can be considered a nominal energy level for the nominal operation of the catheter during treatment of the treatment site.

At step 480, an energy source of the catheter system is adjusted to the conditioning energy level with a source adjuster. In various embodiments, the conditioning energy level can be a predetermined fraction of the nominal energy level (the energizing energy level).

At step 482, an energy multiplexer is scanned across a catheter connector array. In some embodiments, the catheter connector array can be scanned by any suitable scanner known in the art.

At step 484, the source adjuster can be automatically used at some point in time prior to the energizing energy level being applied, the conditioning energy level is applied utilizing the source adjuster. In other words, the source adjuster can automatically adjust the energy source to alternate between the conditioning energy level and the energizing energy level. Alternatively, the source adjuster can be manually initiated by an operator. The energy source delivers energy, which is adjusted by the source adjuster. An energy pulse is sequentially fired into each channel of the catheter. The energy pulse results in energy being delivered equal to the conditioning energy level. In some embodiments, the energy pulse can include one energy pulse or a sequence of energy pulses between greater than 0% of the energizing energy level and less than 100% of the energizing energy level. In various non-exclusive embodiments, the energy pulse can be at least 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99% or less than 99.999% of the energizing energy level or the treatment level.

In some embodiments, the conditioning pulse can be delivered immediately before the energizing pulse instead of firing all low-energy pulses in one pass before the energizing pulse. In such embodiments, the conditioning energy pulse can precede each energizing pulse. In some embodiments, the conditioning energy pulse can be fired with or without inflating the catheter to the inflated state to condition the proximal and/or distal end of one or more of the energy guides. The methods and systems described herein can utilize any suitable combination of energy pulse sequencing options to achieve improved conversion efficiency and overall device performance.

The conditioning pulse or pulses could be fired before or after the energizing pulse. The conditioning pulse could be a low-energy pulse at the end of the energizing pulse that could condition the plasma generator after firing the energizing pulse. The conditioning pulse step can include a continuous chain of small pulses that continuously condition the guide proximal end, the guide distal end, and/or the plasma generator. The energizing pulse can also or alternatively then be fired intermittently between each sequence of small conditioning pulses.

A plurality of coupling areas of a plurality of energy guides can be conditioned with a pretreatment (the conditioning energy level). In some embodiments, when the energy source is a laser, and the energy guide is an optical fiber, the conditioning energy pulse can clear the proximal guide end of the optical fiber from various types of contamination. The conditioning energy pulse or pulses can reduce contamination or particles on the end face of the proximal guide end without damaging the surface of the proximal guide end (e.g., without damaging a glass surface of the energy guide).

The conditioning energy pulse can also reduce the likelihood that contamination vaporizing under high energy would create localized damage that, in turn, could form a pit or a localized defect that would concentrate energy and increase damage. The conditioning energy pulse can further reduce or eliminate the effects of surface contamination, thereby considerably increasing the damage threshold for a lithotripsy device. The conditioning energy pulse can also improve total energy transmission and increase the overall yield of the lithotripsy device.

At or near the distal end of the energy guide(s), the conditioning energy pulse can dislodge or displace micro-bubbles that adhere to the surface of the plasma generator target and limit the energy transmitted onto this surface which reduces the conversion efficiency. In the embodiments where the energy source is a laser, and the energy guide is an optical fiber, the low-energy pulse clears the target from bubbles or other adhering contamination. This approach could also displace other inclusions in the fluid surrounding the plasma generator, which interfere with the transmission of the energy pulse to the plasma generator. These inclusions scatter or absorb energy, thereby limiting conversion efficiency and yield. The conditioning energy pulse would minimize the impact on the plasma-generating mechanism. The conditioning pulse can be fired just before the energy pulse or at any time in between sequential pulses to prevent the buildup of inclusions in the fluid around the plasma generator or on its surfaces.

In some embodiments, the conducted energy is electrons, the energy source is a high-voltage pulse generator, the energy guide is a pair of metallic wires, and the plasma generator is a spark gap located at the distal end of the two wires. The conditioning step can involve firing a low-energy electrical pulse into each plasma circuit to precondition the electrical interface from the console to the wiring. This can reduce oxides and contamination, thereby improving electrical conductivity and energy transmission to the distal plasma generator. The spark gap plasma generators can suffer from the same performance issues as optical plasma generators due to micro-bubbles, particulates, and inclusions. The intermediate conditioning step between treatment cycles would be used to clear these from the spark gap, increasing conductivity, thereby improving the device's conversion efficiency and yield.

At step 486, the balloon of the catheter is positioned and pressurized at a first treatment site. An operator can position the catheter in any suitable location within the patient, including the first treatment site. As used herein, the “first,” “second,” and “third” treatment sites are merely demonstrative for ease of understanding. It is understood that the first treatment site, the second treatment site, and the third treatment site can be the same treatment site or different treatment sites. The surgeon or clinician can position the catheter at any suitable number of treatment sites based on the individual patient's treatment needs. The balloon can also be pressurized with a plasma-generating fluid to prepare the catheter system for treatment.

At step 488, one or more treatment cycles are run at the energizing energy level (also sometimes referred to herein as “full energy”). The catheter system can scan the energy multiplexer across the connector array, firing the energizing energy pulse into each channel through the entire sequence that comprises a treatment cycle. Alternatively, the catheter system can alternate between the conditioning energy level and the energizing energy level for each energy guide or for less than all energy guides.

At step 490, the balloon of the catheter can be deflated and repositioned at a second a treatment site. The catheter can then be re-inflated at the second treatment site, and the same or modified sequence of firing can occur. In some embodiments, the system can scan across the catheter connector array, firing a conditioning pulse sequentially into each catheter channel to dislodge and displace micro-bubbles and other particles. This process preconditions each energy guide and/or plasma generator for improved performance. The catheter system can then be rerun at full energy for one or more treatment cycles at the energizing energy level.

At step 492, depending on the treatment stage and/or treatment needs of the individual patient, the method can repeat steps 482 through 490 until the treatment is complete. Additionally, depending on the conditioning needs of the light guide, the interior of the inflatable balloon, and/or the plasma generator, steps 482 and 484 can be repeated until the conditioning of each of the light guide, the interior of the inflatable balloon, and/or the plasma generator is complete.

### Lasers

The lasers suitable for use herein can include various types of lasers, including lasers and lamps. Suitable lasers can include short pulse lasers on the sub-millisecond timescale. In some embodiments, the laser can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths, and energy levels that can be employed to achieve plasma in the balloon fluid of the catheters illustrated and/or described herein. In various embodiments, the pulse widths can include those falling within a range including from at least 10 ns to 200 ns. In some embodiments, the pulse widths can include those falling within a range including from at least 20 ns to 100 ns. In other embodiments, the pulse widths can include those falling within a range including from at least 1 ns to 5000 ns.

Exemplary nanosecond lasers can include those within the UV to IR spectrum, spanning wavelengths of about 10 nanometers to 1 millimeter. In some embodiments, the lasers suitable for use in the catheter systems herein can include those capable of producing light at wavelengths of from at least 750 nm to 2000 nm. In some embodiments, the lasers can include those capable of producing light at wavelengths of from at least 700 nm to 3000 nm. In some embodiments, the lasers can include those capable of producing light at wavelengths of from at least 100 nm to 10 micrometers (μm). Nanosecond lasers can include those having repetition rates of up to 200 kHz. In some embodiments, the laser can include a Q-switched thulium:yttrium-aluminum-garnet (Tm:YAG) laser. In some embodiments, the laser can include a neodymium:yttrium-aluminum-garnet (Nd:YAG), holmium:yttrium-aluminum-garnet (Ho:YAG), erbium:yttrium-aluminum-garnet (Er:YAG), excimer laser, helium-neon laser, carbon dioxide laser, as well as doped, pulsed, fiber lasers.

### Pressure Waves

The catheters illustrated and/or described herein can generate pressure waves having maximum pressures in the range of at least 1 megapascal (MPa) to 100 MPa. The maximum pressure generated by a particular catheter will depend on the laser, the absorbing material, the bubble expansion, the propagation medium, the balloon material, and other factors. In some embodiments, the catheters illustrated and/or described herein can generate pressure waves having maximum pressures in the range of at least 2 MPa to 50 MPa. In other embodiments, the catheters illustrated and/or described herein can generate pressure waves having maximum pressures in the range of at least 2 MPa to 30 MPa. In yet other embodiments, the catheters illustrated and/or described herein can generate pressure waves having maximum pressures in the range of at least 15 MPa to 25 MPa. In some embodiments, the catheters illustrated and/or described herein can generate pressure waves having peak pressures of greater than or equal to 1 MPa, 2 MPa, 3 MPa, 4 MPa, 5 MPa, 6 MPa, 7 MPa, 8 MPa, 9 MPa, 10 MPa, 11 MPa, 12 MPa, 13 MPa, 14 MPa, 15 MPa, 16 MPa, 17 MPa, 18 MPa, 19 MPa, 20 MPa, 21 MPa, 22 MPa, 23 MPa, 24 MPa, or 25 MPa, 26 MPa, 27 MPa, 28 MPa, 29 MPa, 30 MPa, 31 MPa, 32 MPa, 33 MPa, 34 MPa, 35 MPa, 36 MPa, 37 MPa, 38 MPa, 39 MPa, 40 MPa, 41 MPa, 42 MPa, 43 MPa, 44 MPa, 45 MPa, 46 MPa, 47 MPa, 48 MPa, 49 MPa, or 50 MPa. It is appreciated that the catheters illustrated and/or described herein can generate pressure waves having operating pressures or maximum pressures that can fall within a range, wherein any of the foregoing numbers can serve as the lower or upper bound of the range, provided that the lower bound of the range is a value less than the upper bound of the range.

Therapeutic treatment can act via a fatigue mechanism or a brute force mechanism. Pressures between the extreme ends of these two ranges may act upon a treatment site using a combination of a fatigue mechanism and a brute force mechanism. For a fatigue mechanism, operating pressures would be about at least 0.5 MPa to 2 MPa, or about 1 MPa. For a brute force mechanism, operating pressures would be about at least 20 MPa to 30 MPa, or about 25 MPa.

The pressure waves described herein can be imparted upon the treatment site from a distance within a range from at least 0.01 millimeters (mm) to 25 mm, extending radially from a longitudinal axis of a catheter placed at a treatment site. In some embodiments, the pressure waves can be imparted upon the treatment site from a distance within a range from at least 1 mm to 20 mm, extending radially from a longitudinal axis of a catheter placed at a treatment site. In other embodiments, the pressure waves can be imparted upon the treatment site from a distance within a range from at least 0.1 mm to 10 mm, extending radially from a longitudinal axis of a catheter placed at a treatment site. In yet other embodiments, the pressure waves can be imparted upon the treatment site from a distance within a range from at least 1.5 mm to 4 mm, extending radially from a longitudinal axis of a catheter placed at a treatment site. In some embodiments, the pressure waves can be imparted upon the treatment site from a range of at least 2 MPa to 30 MPa at a distance from 0.1 mm to 10 mm. In some embodiments, the pressure waves can be imparted upon the treatment site from a range of at least 2 MPa to 25 MPa at a distance from 0.1 mm to 10 mm. In some embodiments, the pressure waves can be imparted upon the treatment site from a distance that can be greater than or equal to 0.1 mm, 0.2 mm, 0.3 mm, 0.4 mm, 0.5 mm, 0.6 mm, 0.7 mm, 0.8 mm, or 0.9 mm, 1 mm, 2 mm, 3 mm, 4 mm, 5 mm, 6 mm, 7 mm, 8 mm, 9 mm, or 10 mm, or can be an amount falling within a range between, or outside the range of any of the foregoing.

By shaping the temporal form of the optical pulse to have a fast rise time and minimal overshoot (ideally approaching a square wave), the efficiency for generating the pressure wave can be improved, and the amount of energy that can be delivered in a given time interval can be increased while decreasing the peak laser intensity to remain below the damage threshold of the optical fiber.

As described in detail herein, in various embodiments, the present technology can be utilized to address problems that exist in more conventional catheter systems or any future catheter systems that are intended to generate a plasma for use during treatment. For example, the present technology can:

1) Reduce the effect of micro-bubbles or other inclusions in the plasma generator;

2) Improve the damage threshold of the energy transmission medium or materials;

3) Improve energy conversion in the plasma generator and overall yield of the catheter system;

4) Enable preconditioning of the energy guide and/or the plasma generator using gating and/or pulse conditioning; and

5) Address physical and material limitations that impact the performance and reliability of a lithotripsy device that utilizes an energy source to create a plasma, producing a localized, high-energy mechanical effect.

It should be noted that, as used in this specification and the appended claims, the singular forms “a,” “an,” and “the” include plural referents unless the content and/or context clearly dictates otherwise. It should also be noted that the term “or” is generally employed in its sense, including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

As used herein, the recitation of numerical ranges by endpoints shall include all numbers subsumed within that range, inclusive (e.g., 2 to 8 includes 2, 2.1, 2.8, 5.3, 7, 8, etc.).

It is recognized that the figures shown and described are not necessarily drawn to scale, and that they are provided for ease of reference and understanding, and for relative positioning of the structures.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. A method for conditioning one of an energy guide and a plasma generator in a catheter, the method comprising the steps of:
positioning a plasma generator within an inflatable balloon near a distal end of an energy guide;
coupling a proximal end of the energy guide to an energy source;
positioning the inflatable balloon adjacent to a treatment site;
adjusting the energy source with a source adjuster to generate (a) a conditioning pulse having a conditioning energy level, and (b) an energizing pulse having an energizing energy level, the conditioning energy level being lower than the energizing energy level; and
alternately delivering the conditioning pulse and the energizing pulse to the proximal end of the energy guide to condition one of (i) the proximal end of the energy guide, and (ii) the plasma generator, to generate a plasma within the inflatable balloon.

2. The method of claim 1 wherein the step of coupling includes optically coupling the proximal end of the energy guide to the energy source.

3. The method of claim 1 wherein the step of coupling includes electrically coupling the proximal end of the energy guide to the energy source.

4. The method of claim 1 wherein the source adjuster automatically adjusts the energy source to alternate between the conditioning energy level and the energizing energy level.

5. The method of claim 4 wherein the source adjuster adjusts the energy source to alternate between the conditioning energy level and the energizing energy level after a predetermined number of conditioning pulses.

6. The method of claim 1 wherein the catheter includes a plurality of energy guides each having a proximal end and a distal end, each distal end being positioned near a respective spaced-apart plasma generator within the inflatable balloon, and the steps of adjusting and alternately delivering are implemented for each of the plurality of energy guides.

7. The method of claim 6 wherein the conditioning energy level is applied to the proximal end of each of the plurality of energy guides before the energizing energy level is applied to the proximal end of each of the plurality of energy guides.

8. The method of claim 6 wherein the conditioning energy level and the energizing energy level are applied to the proximal end of each of the plurality of energy guides in an alternating manner.

9. The method of claim 6 wherein the source adjuster alternates from the conditioning energy level to the energizing energy level after a predetermined number of conditioning pulses.

10. The method of claim 1 wherein the energy guide includes an optical fiber and the energy source includes a laser.

11. The method of claim 1 wherein the energy guide includes a pair of metallic wires and the energy source includes a high-voltage energy generator.

12. The method of claim 1 wherein the step of positioning the plasma generator includes the plasma generator including a metallic plasma target that receives energy via the energy guide.

13. A catheter system for generating a plasma within an inflatable balloon, the catheter system comprising:
an energy guide that is configured to selectively receive energy, the energy guide including a guide proximal end and a guide distal end;
an inflatable balloon;
a plasma generator that is positioned within the balloon near the guide distal end of the energy guide; and
an energy source that is coupled to the guide proximal end of the energy guide, the energy source including a source adjuster that is configured to alternately generate an energizing pulse and a conditioning pulse having a lower energy level than the energizing pulse, the conditioning pulse conditioning one of (a) the guide proximal end of the energy guide, and (b) the plasma generator.

14. The catheter system of claim 13 wherein the energy guide is optically coupled to the energy source.

15. The catheter system of claim 13 wherein the energy guide is electrically coupled to the energy source.

16. The catheter system of claim 13 wherein the source adjuster is configured to automatically alternately adjust the energy source between the conditioning energy level and the energizing energy level.

17. The catheter system of claim 16 wherein the source adjuster is configured to adjust the energy source to alternate between the conditioning energy level and the energizing energy level after a predetermined number of conditioning pulses.

18. The catheter system of claim 13 wherein the energy guide includes an optical fiber and the energy source includes a laser.

19. The catheter system of claim 13 wherein the energy guide includes a pair of metallic wires and the energy source includes a high-voltage energy generator.

20. The catheter system of claim 13 wherein the plasma generator includes a metallic plasma target that receives energy via the energy guide.
